(19) United States
(12) Patent Application Publication
(10) Pub. No.: US 2019/0046548 A1
(43) Pub. Date: Feb. 14, 2019

(54) Formulation and Manufacturing Method for Color-Changing Eyedrops

(71) Applicant: iCOLOUR LLC, Houston, TX (US)

(72) Inventor: Jose Cervantes, Houston, TX (US)

(21) Appl. No.: 16/059,032

(22) Filed: Aug. 8, 2018

Related U.S. Application Data
(60) Provisional application No. 62/542,698, filed on Aug. 8, 2017.

(51) Int. Cl. A61K 31/7008; A61K 33/14; A61K 33/22

(52) U.S. Cl. CPC A61K 31/7008; A61K 33/22; A61K 33/14

(57) ABSTRACT

Disclosed is a formulation and a manufacturing method for color-changing eyedrops.

The current application claims a priority to the U.S. Provisional Patent application Ser. No. 62/542,698 filed on Aug. 8, 2017.

### FIELD OF THE INVENTION

The present invention generally relates to cosmetic products. More specifically, the present invention is a substance designed to temporarily change the natural color of the eye.

### BACKGROUND OF THE INVENTION

People are born with various physical characteristics, that are genetically inherited from their parents. Often times, people want to change their physical appearance. Eye color is a genetically inherited, and generally speaking cannot be easily changed. Ophthalmology is a specialized area of medicine that focuses with the anatomy of the eye. As technology advances, numerous solutions have been developed to help assist users with correcting vision problems. Procedures range from accessories as simple as eyeglasses, to extensive surgical procedures. Age related factors are some of the biggest contributors to vision problems. As a result, contact lenses are commonly used by people to correct visions problems. In addition, some contacts lenses can be used to change the appearance of eye color. Conventional contact lenses are manufactured out of a water absorbing material known as hydrogel. Tinted dyes may be added to the material, if the user decides to alter appearance of their eye color.

The cosmetics industry is a billion-dollar industry that focuses on helping people maintain or change their appearance in various ways. Eye drops have numerous medical uses. Most eye problems, have a matching eye-drop treatment, that can alleviate some of the symptoms based on each condition. Eye drops are generally designed to serve a practical purpose with no esthetical function.

Wearing contact lenses for aesthetic purposes, is not uncommon however, in the Unites States the use of contact lenses requires a prescription. In addition, it is not uncommon for users to experience discomfort, and numerous side effects. The present invention aims to solve some of the problems mentioned above, by disclosing an eye drop solutions that allows users to change the color of the iris, for an extended period of time.

### BRIEF DESCRIPTION OF THE DRAWINGS

FIGS. 1 through 3 illustrate a formulation for color-changing eyedrops. FIGS. 4 through 10 illustrate a formulation for color-changing eyedrops.

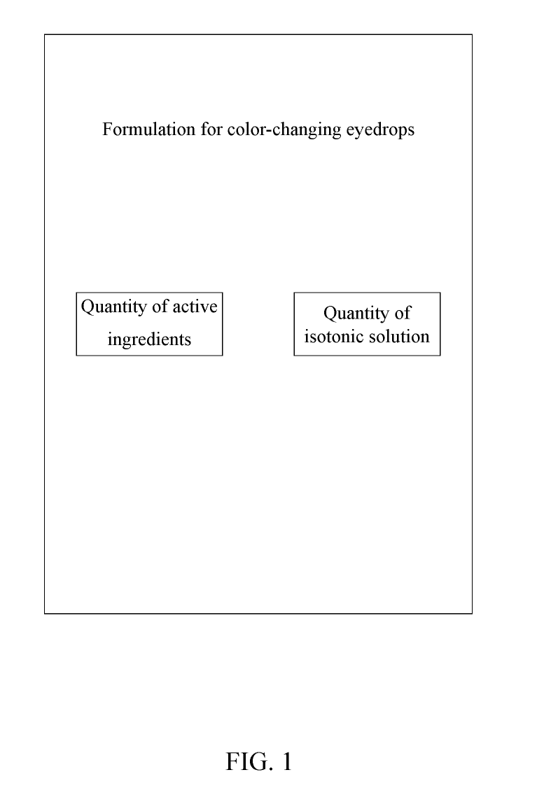

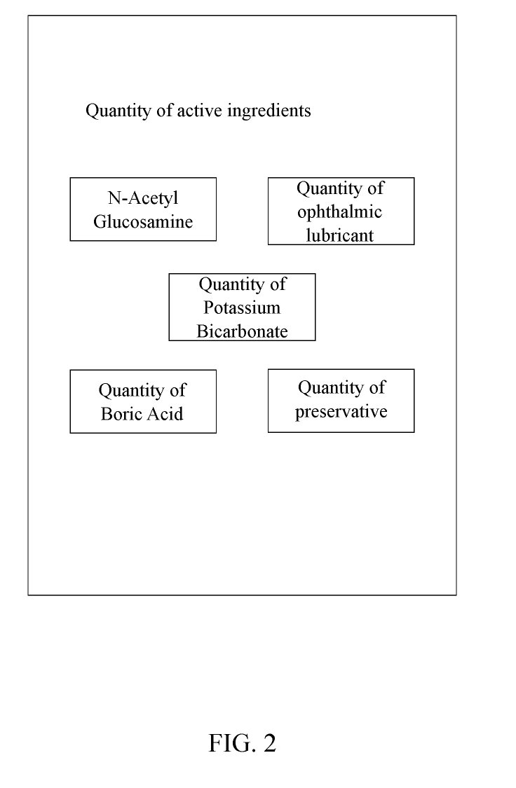

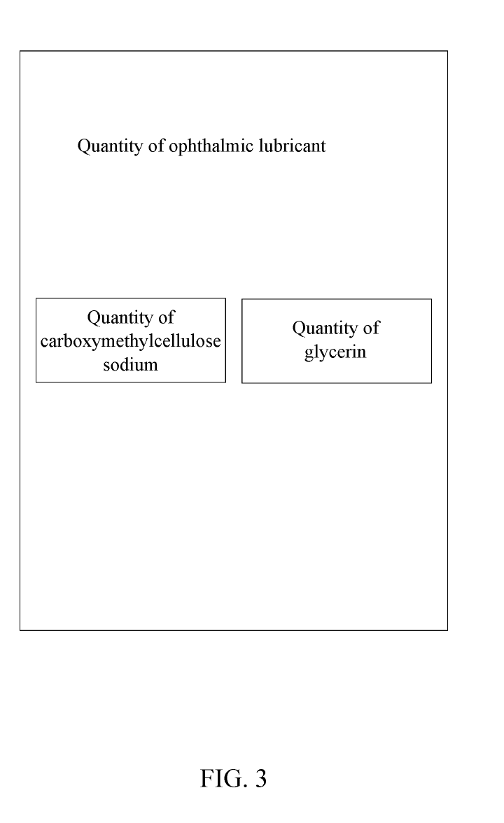

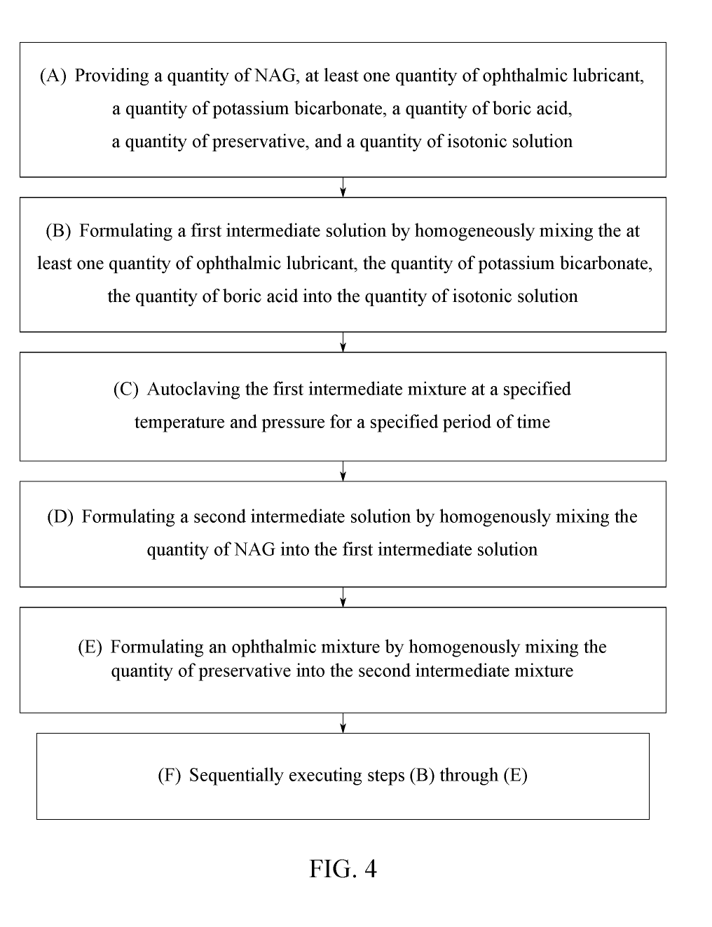

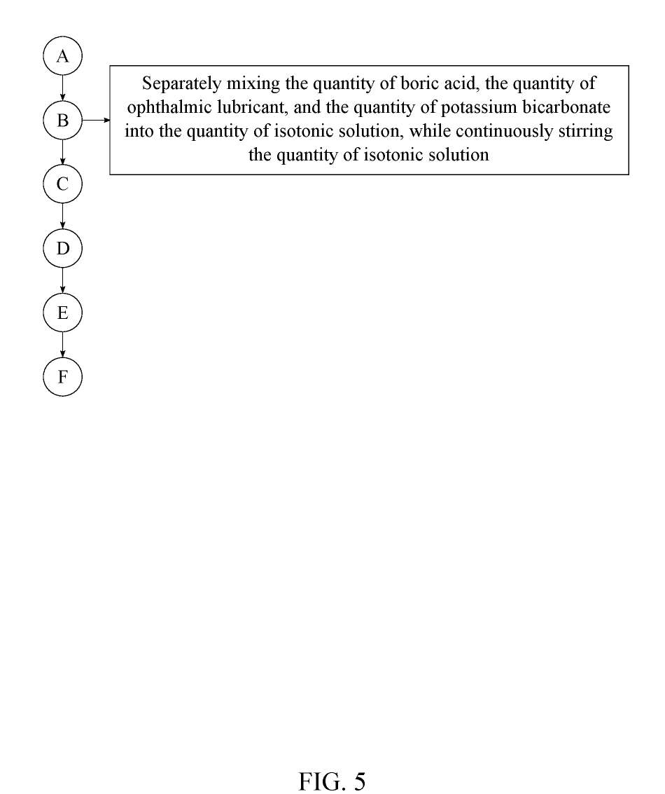

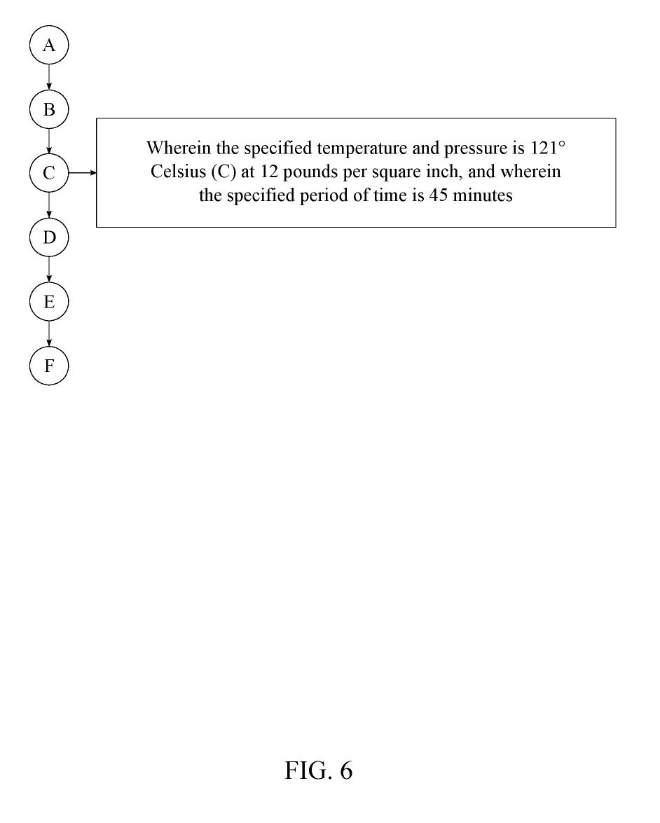

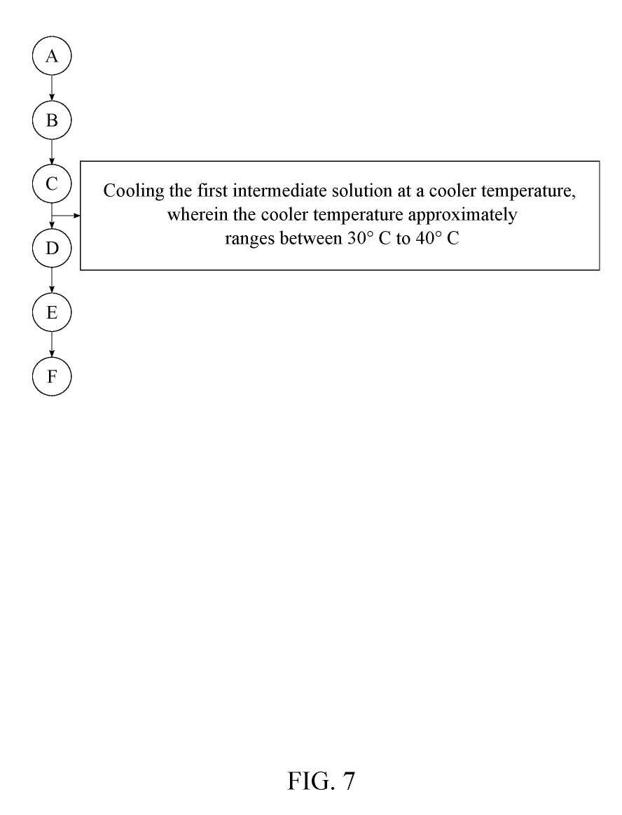

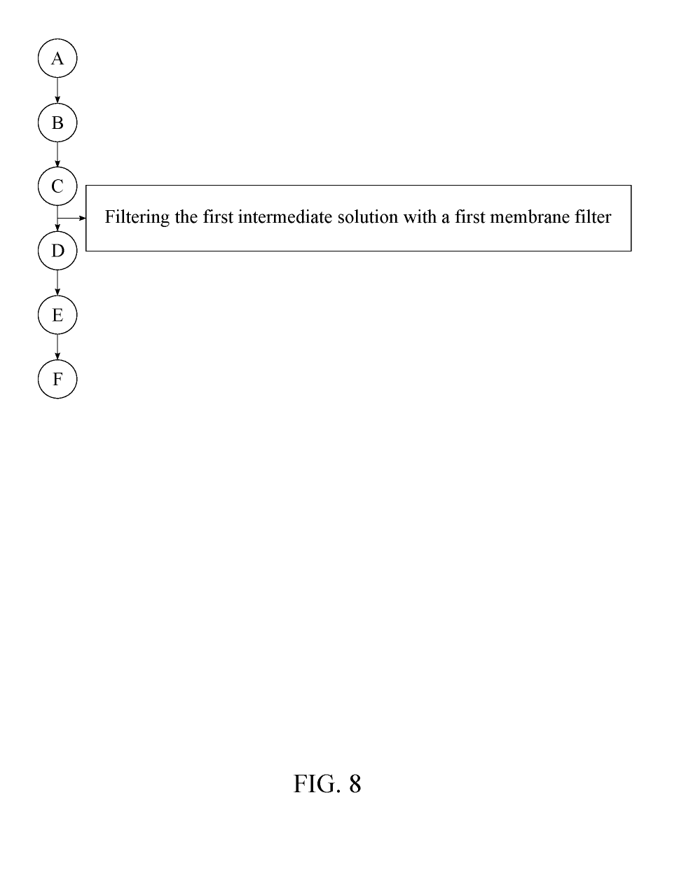

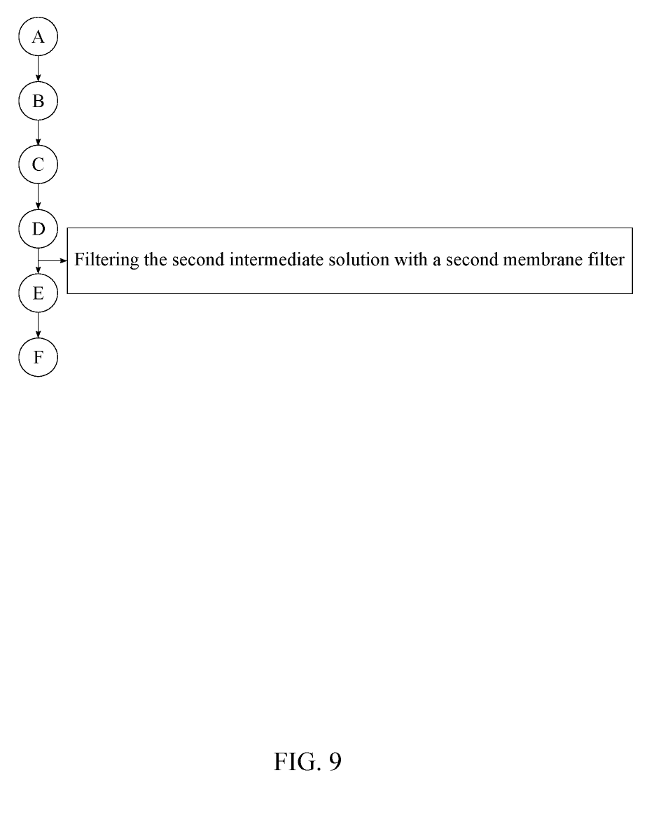

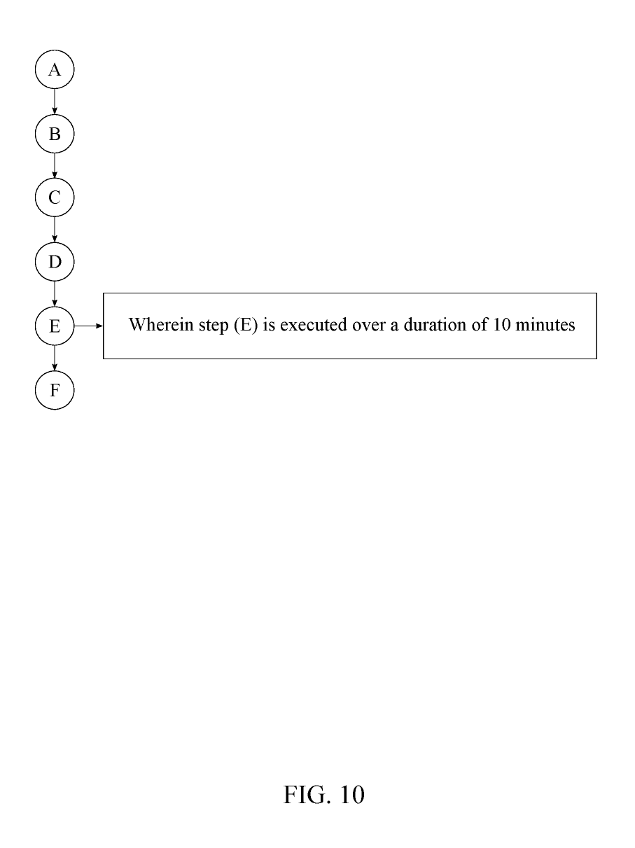

### DETAILED DESCRIPTION OF THE INVENTION

All illustrations of the drawings are for the purpose of describing selected versions of the present invention and are not intended to limit the scope of the present invention.

The present invention is a formulation for color-changing eyedrops. The present invention allows a user to gradually change their eye color to a color of their choice, over a period of time. The present invention comprises a quantity of active ingredients and a quantity of isotonic solution. The quantity of active ingredients is the main compositional constituents that change the concentration of melanin in the iris of an eye. The quantity of isotonic solution is used as a base solution to convey the quantity of active ingredients across the iris of an eye. Thus, the quantity of active ingredients and the quantity of isotonic solution needs to be homogeneously mixed into an ophthalmic mixture, which is final applyable form of the present invention. More specifically, the quantity of active ingredients approximately ranges between 4.1 weight percent (wt. %) to 5.1 wt. % of the ophthalmic mixture, which prevent overdosing the user's eye with the quantity of active ingredients. In addition, the quantity of isotonic solution is preferably sterile water and has a pH level of 6.4. However, an acceptable pH range for the quantity of isotonic solution is between 6.3 to 6.5.

As the present invention is absorbed through the ocular surface, the quantity of active ingredients begins to block the production of melanin, by inhibiting the activation of the enzyme required to produce melanin. The amount of change in the eye color is proportionate to the amount of melanin that is reaching the iris, which how the present invention changes the eye color. Moreover, the quantity of active ingredients comprises a quantity of N-Acetyl Glucosamine (NAG), at least one quantity of ophthalmic lubricant, a quantity of potassium bicarbonate, a quantity of boric acid, and a quantity of preservative. The quantity of NAG is the actual compositional constituent that is used to inhibit the production of melanin in the iris. The at least one ophthalmic lubricant allows the eyelid to easily slide across the ocular surface as the ophthalmic mixture is applied across a user's eye. The at least one quantity of ophthalmic lubricant preferably comprises a quantity of carboxymethylcellulose sodium and a quantity of glycerin. The quantity of preservative is used to extend the shelf life of the ophthalmic mixture. The quantity of preservative is preferably purified benzyl alcohol.

A preferred embodiment of the present invention uses the following compositional ratios between the quantity of active ingredients in order to efficiently and effectively change the color of the iris for an eye. The quantity of NAG approximately ranges between 24.3 wt. % to 39.1 wt. % of the quantity of active ingredients. The quantity of NAG is directly proportional to the color change in the iris of an eye, and, thus, the amount of NAG needs to be added to the ophthalmic mixture is dependent on the exact color that a user wants to change their eye color. In regards to the at least one ophthalmic lubricant, the quantity of carboxymethylcellulose sodium approximately ranges between 5.9 wt. % to 7.3 wt. % of the quantity of active ingredients, and quantity of glycerin approximately ranges between 19.6 wt. % to 24.3 wt. % of the quantity of active ingredients. Furthermore, the quantity of potassium bicarbonate approximately ranges between 19.6 wt. % to 24.3 wt. % of the quantity of active ingredients. In addition, the quantity of boric acid approximately ranges between 9.8 wt. % to 12.2 wt. % of the quantity of active ingredients. Moreover, the quantity of preservative approximately ranges between 6.1 wt. % to 7.6 wt. % of the quantity of active ingredients. However, the present invention is not limited to these compositional ratios. Slight variations to those compositional ratios may be done to the ophthalmic mixture to better suit each individual needs and requirements of each user.

The present invention is also a method of manufacturing color-changing eyedrops. The overall process followed by the manufacturing method allows the present invention to formulate the ophthalmic mixture in accordance to the aforementioned compositional ratios. The overall process begins by being provided with a quantity of NAG, at least one quantity of ophthalmic lubricant, a quantity of potassium bicarbonate, a quantity of boric acid, a quantity of preservative, and a quantity of isotonic solution (Step A). The overall process continues by formulating a first intermediate solution by homogeneously mixing the at least one quantity of ophthalmic lubricant, the quantity of potassium bicarbonate, the quantity of boric acid into the quantity of isotonic solution (Step B). More specifically, the quantity of boric acid, the quantity of ophthalmic lubricant, and the quantity of potassium bicarbonate are separately mixed in the quantity of isotonic solution, one by one, while continuously stirring the quantity of isotonic solution. The overall process continues by autoclaving the first intermediate mixture at a specified temperature and pressure for a specified period of time (Step C). The specified temperature and pressure are preferably 121° Celsius (C) at 12 pounds per square inch, and the specified period of time is preferably 45 minutes. The first intermediate solution could then be cooled at a cooler temperature than the temperature specified in Step C. The cooler temperature would approximately range between 30° C. to 40° C.

Once the first intermediate solution has been processed, the overall process continues by formulating a second intermediate solution by homogeneously mixing the quantity of NAG into the first intermediate solution (Step D), which is when the proper amount of NAG is selected to result in a corresponding eye color. The overall process concludes the ophthalmic mixture by formulating by homogeneously mixing the quantity of preservative into the second intermediate mixture (Step E), which is preferably executed for a duration of 10 minutes. Moreover, Steps B through E are sequentially executed during the overall process.

The manufacturing method can also use filtration to remove particulate that is too large to make contact with the ocular surface. Thus, a first membrane filter is used to filter the first intermediate solution between Step C and Step D. The first membrane filter is preferably a 0.45 micron NN type 293 mm diameter. In addition, a second membrane filter is used to filter the second intermediate solution between Step D and Step E. The second membrane filter is preferably a 0.45 micron NN type 293 mm diameter.

An optional final step is to check the pH level of the ophthalmic mixture. The lower limit of the pH level is 6.3, while the maximum level is 6.5. Once all the required testing is completed, the resulting solution may be packaged in an aseptic environment.

The formulation is in liquid form and can be applied to the user's eye directly. In addition, the solution and the container in which it is stored, must be sterile to ensure a safe application, and to prevent the spread of bacteria that may cause eye disorders. The present invention provides a gradual inhibitor of melanin, thus altering the color of the user's eye. Numerous variations are available, depending on the color the user desires to ultimately achieve.

Eye color is a genetic trait determined by the pigmentation of the eye's iris, and the frequency dependence of the scattering of the light in the stroma of the iris. In humans, the pigmentation of the eye varies from brown to black. Other eyes may appear, blue or green in color, from the scattering of light in the stroma. As people age, it is not uncommon for the eyes to change color. Discoloration and changes in opacity, are a common occurrence as people advance in age.

Although the invention has been explained in relation to its preferred embodiment, it is to be understood that many other possible modifications and variations can be made without departing from the spirit and scope of the invention as hereinafter claimed.

What is claimed is:

1. A formulation for color-changing eyedrops comprises:
a quantity of active ingredients;
a quantity of isotonic solution;
the quantity of active ingredients comprises a quantity of N-Acetyl Glucosamine (NAG), at least one quantity of ophthalmic lubricant, a quantity of potassium bicarbonate, a quantity of boric acid, and a quantity of preservative; and
the quantity of active ingredients and the quantity of isotonic solution being homogeneously mixed into an ophthalmic mixture.

2. The formulation for color-changing eyedrops as claimed in claim 1, wherein the quantity of active ingredients approximately ranges between 4.1 weight percent (wt. %) to 5.1 wt. % of the ophthalmic mixture.

3. The formulation for color-changing eyedrops as claimed in claim 1, wherein the quantity of isotonic solution is sterile water.

4. The formulation for color-changing eyedrops as claimed in claim 3, wherein a pH level of the quantity of isotonic solution is approximately 6.4.

5. The formulation for color-changing eyedrops as claimed in claim 1, wherein the quantity of NAG approximately ranges between 24.3 wt. % to 39.1 wt. % of the quantity of active ingredients.

6. The formulation for color-changing eyedrops as claimed in claim 1 comprises:
the at least one quantity of ophthalmic lubricant comprises a quantity of carboxymethylcellulose sodium; and
the quantity of carboxymethylcellulose sodium approximately ranging between 5.9 wt. % to 7.3 wt. % of the quantity of active ingredients.

7. The formulation for color-changing eyedrops as claimed in claim 1 comprises:
the at least one quantity of ophthalmic lubricant comprises a quantity of glycerin; and
the quantity of glycerin approximately ranging between 19.6 wt. % to 24.3 wt. % of the quantity of active ingredients.

8. The formulation for color-changing eyedrops as claimed in claim 1, wherein the quantity of potassium bicarbonate approximately ranges between 19.6 wt. % to 24.3 wt. % of the quantity of active ingredients.

9. The formulation for color-changing eyedrops as claimed in claim 1, wherein the quantity of boric acid approximately ranges between 9.8 wt. % to 12.2 wt. % of the quantity of active ingredients.

10. The formulation for color-changing eyedrops as claimed in claim 1 comprises:
the quantity of preservative being purified benzyl alcohol; and
the quantity of preservative approximately ranging between 6.1 wt. % to 7.6 wt. % of the quantity of active ingredients.

11. A method of manufacturing color-changing eyedrops, the method comprises the steps of:
(A) providing a quantity of NAG, at least one quantity of ophthalmic lubricant, a quantity of potassium bicarbonate, a quantity of boric acid, a quantity of preservative, and a quantity of isotonic solution;
(B) formulating a first intermediate solution by homogeneously mixing the at least one quantity of ophthalmic lubricant, the quantity of potassium bicarbonate, the quantity of boric acid into the quantity of isotonic solution;
(C) autoclaving the first intermediate mixture at a specified temperature and pressure for a specified period of time;
(D) formulating a second intermediate solution by homogeneously mixing the quantity of NAG into the first intermediate solution; and
(E) formulating an ophthalmic mixture by homogeneously mixing the quantity of preservative into the second intermediate mixture;
(F) sequentially executing steps (B) through (E).

12. The method of manufacturing color-changing eyedrops, the method as claimed in claim 11 comprises the step of:
separately mixing the quantity of boric acid, the quantity of ophthalmic lubricant, and the quantity of potassium bicarbonate into the quantity of isotonic solution, while continuously stirring the quantity of isotonic solution during step (B).

13. The method of manufacturing color-changing eyedrops, the method as claimed in claim 11, wherein the specified temperature and pressure are 121° Celsius (C) at 12 pounds per square inch, and wherein the specified period of time is 45 minutes.

14. The method of manufacturing color-changing eyedrops, the method as claimed in claim 11 comprises the step of:
cooling the first intermediate solution at a cooler temperature after step (C), wherein the cooler temperature approximately ranges between 30° C. to 40° C.

15. The method of manufacturing color-changing eyedrops, the method as claimed in claim 11 comprises the step of:
filtering the first intermediate solution with a first membrane filter in between steps (C) and (D).

16. The method of manufacturing color-changing eyedrops, the method as claimed in claim 15, wherein the first membrane filter is a 0.45 micron NN type 293 mm diameter.

17. The method of manufacturing color-changing eyedrops, the method as claimed in claim 11 comprises the step of:
filtering the second intermediate solution with a second membrane filter in between steps (D) and (E).

18. The method of manufacturing color-changing eyedrops, the method as claimed in claim 17, wherein the first membrane filter is a 0.22 micron NN type 293 mm diameter.

19. The method of manufacturing color-changing eyedrops, the method as claimed in claim 11, wherein the step (E) is executed over a duration of 10 minutes.
